(19) United States
(12) Patent Application Publication
(10) Pub. No.: US 2025/0186397 A1
(43) Pub. Date: Jun. 12, 2025

(54) COMPOUNDS FOR THE USE IN THE TREATMENT OF MOOD DISORDERS

(71) Applicant: Kanna Health Ltd, Kent (GB)

(72) Inventors: Royston Anthony GRAY, Broxted (GB); Michelle KOKKINOU, London (GB); Thomas PFEIFFER, London (GB)

(21) Appl. No.: 18/843,177

(22) PCT Filed: Mar. 2, 2023
(86) PCT No.: PCT/GB2023/050494

§ 371 Date: Aug. 30, 2024

Related U.S. Application Data
(60) Provisional application No. 63/315,662, filed on Mar. 2, 2022.

(30) Foreign Application Priority Data
Jan. 9, 2023 (GB) 2300302.3

(51) Int. Cl. A61K 31/404; A61P 25/22; A61P 25/24

(52) U.S. Cl. CPC A61K 31/404; A61P 25/22; A61P 25/24

(57) ABSTRACT

The present invention relates to the use of mesembrine alkaloids for use in the treatment of mood disorders. In particular, the invention provides compositions and methods for improving symptoms associated with anxiety and/or mood disorders, or a disorder where anxiety and/or depression are comorbid.

The present application is related to, and claims the benefit of U.S. 63/315,662, filed on Mar. 2, 2022 (02.03.2022), the contents of which are hereby incorporated by reference in their entirety. It further is related to and claims the benefit of GB 2300302.3, filed on Jan. 9, 2023 (09.01.2023), the contents of which are hereby incorporated by reference in their entirety.

### FIELD OF THE INVENTION

The present invention relates to the use of mesembrine alkaloids for use in the treatment of mood disorders. In particular, the invention provides compositions and methods for improving symptoms associated with anxiety and/or mood disorders, or a disorder where anxiety and/or depression are comorbid.

### BACKGROUND TO THE INVENTION

The prevalence of mood disorders is estimated at 9.7% of U.S. adults. The prevalence of any mood disorder among adults is higher for females (11.6%) than for males (7.7%). An estimated 21.4% of U.S. adults experience any mood disorder at some time in their lives.

Current treatments for mood disorders often consist of a combination of psychotherapy and one or more daily medications that regulate neurotransmitters such as dopamine, serotonin, and norepinephrine. These medications often take weeks to months to achieve their full effects and in the meantime, individuals continue to suffer from their symptoms and be at risk of self-harm, as well as harm to their personal and professional lives.

Mood disorders (as defined by DSM-IV) or Bipolar disorders and Depressive disorders (as defined by DSM-V) include major depressive disorder (MDD) or clinical depression; bipolar I disorder or manic depression; bipolar II disorder or hypomania; cyclothymic disorder; bipolar and related disorder due to another medical condition; depressive disorder due to another medical condition; substance or medication induced bipolar disorder; substance or medication induced depressive disorder; disruptive mood dysregulation disorder; persistent depressive disorder; and premenstrual dysphoric disorder. Similarly, to anxiety disorders the treatment of mood disorders usually involves a combination of CBT and medications such as antidepressants.

There is a major impact of anxiety and mood disorders. For example, around 90% of those who die from suicide have an underlying mental illness and people with an anxiety or mood disorder have a higher mortality rate and die an average of 8 years earlier than those without such conditions.

Research shows that approximately 9.5% of US adults have been diagnosed with an anxiety or mood disorder with women more than twice as likely to suffer than men.

In the UK 25% of people will experience a mental health problem of some kind each year with 1 in 15 people attempting suicide at some point in their life.

There is a clear unmet need for prevention or treatment of anxiety and/or mood disorders.

Mesembrine alkaloids naturally occur in the Mesembryanthemum tortuosum L (previously referred to as Sceletium tortuosum) species of plants indigenous to South Africa. The genus of plant, classified under the Aizoaceae family, is indigenous to the Western, Eastern and Northern Cape province of South Africa. Extracts of M. tortuosum comprise a mixture of many different alkaloid compounds including mesembrenol, Δ7mesembrenone, mesembrine mesembranol, mesembrenone, and epimesembranol.

Extracts of M. tortuosum have a long history of use in traditional medicine by the San and Khoikhoi people in South Africa where it was used as a masticatory and a medicine to quench their thirst, fight fatigue and for healing, social, and spiritual purposes.

More recently studies have revealed that extracts of the plant have numerous biological properties and extracts of M. tortuosum may be useful in the treatment of anxiety and depression, psychological and psychiatric disorders, improving mood, promoting relaxation and happiness.

An in vivo study in rats demonstrated a positive effect of a whole plant extract of M. tortuosum on restraint-induced anxiety (Smith, 2011), and a small series of case reports described preliminary evidence for antidepressant and anxiolytic activity in patients suffering from major depression who were treated with tablets comprising a standardized extract of milled M. tortuosum raw material (Gericke, 2001). A dietary supplement comprising such material is available as Zembrin®. However, it is not known whether the whole plant extract is required for the effect to occur as has been shown in some naturally derived medications, for example Sativex® (Jazz Pharmaceuticals), or indeed whether there are one or more specific alkaloids within the plant extract that is responsible for the activity.

Clearly there are numerous problems associated with the use of a whole plant extract as it is very difficult to standardise the components of the extract to ensure quality, safety and efficacy of the extract. Furthermore, it is expensive to grow plants then extract them, particularly if the plant extract is to then be used as a medication as all parts of the growing and extraction process need to be tightly controlled and regulated. Additionally relying on crops grown outdoors means extremes of weather conditions such as drought or temperature fluctuations can seriously impair the yield and composition of the plant components.

A study published in 2022 (Olatunji et al., 2022) identified 25 different alkaloids from M. tortuosum which were grouped into four distinct structural classes. However, at least 30 different mesembrine alkaloids are known to exist, these include: mesembrine, mesembrenone, Δ7mesembrenone, 4′-O-demethylmesembranol, mesembrenol, 4′-O-demethylmesembrenol, mesembranol, 4′-O-demethylmesembrenone, sceletenone, N-demethly-N-formulmesembrenone, O-acetylmesembrenol, mesembrane, N-demethelymesembrenol, N-demethylmesembranol, hordenine, joubertiamine, dehydrojoubertiamine, dehydrojoubertiamine, joubertinamine, O-methyldehydrojoubertiamine, O-methyljoubertiamine, O-methyldihydrojoubertiamine, 3′-methoxy-4′-O-methyljoubertiamine, 3′-methoxy-4′O-methyljoubertiaminol, 4-(3,4-dimethoxyphenyl-4-[2-acetylmethylamino)ethyl]cyclohexanone, 4-(3-methoxy-4-hydroxy-phenyl)-4-[2-acetylmethylamino)ethyl]cyclohexadienone, sceletium alkaloid A-4, tortuosamine, N-formultortuosamine, and N-acetyltotuosamine.

A study published in 2012 looked at the chemotypic variation in M. tortuosum and products that comprise extracts from the plant. There was a wide degree of variability in the mesembrine alkaloid content of the 151 specimens examined. The total alkaloid content of wild plant material was found to vary between 0.11 and 1.99% of dry weight. Five main chemotypes were identified. Chemotype A was devoid of mesembrine-type alkaloids. Chemotypes B, C and E were characterised by high levels of mesembrenol (64.96-95.55%), mesembrine (51.25-92.50%) and mesembrenone (50.86-72.51%), respectively, whereas Chemotype D contained all four alkaloids in moderate amounts (Shikanga et al., 2012).

The patent U.S. Pat. No. 9,381,220 describes a composition comprising S. tortuosum extract which comprises the alkaloids mesembrenol and mesembrenone at greater than 80% of the total extract being a dual serotonin-uptake inhibitor and a PDE4 inhibitor. The data in the patent relies on the selection of plant species which comprise a specific composition of mesembrine alkaloids.

A study published in January 2022 (Maphanga et al., 2022) isolated and tested four mesembrine alkaloids, mesembrine, mesembrenone, mesembrenol and mesembranol in a zebrafish model of anxiety. All alkaloids demonstrated an anxiolytic-like effect; however, mesembrenone and mesembranol demonstrated greater anxiolytic-like activity than mesembrine and mesembrenol.

Until now the effects of specific isolated mesembrine alkaloids have not been studied in in vivo mammal models of mood disorders, however the applicant has surprisingly shown that specific isolated mesembrine alkaloids are able to reduce or ameliorate anxiety and mood disorders in animal models of anxiety and depression. Contrary to the finding by Maphanga et al. the alkaloid mesembrenol was found to display a better anxiolytic effect than other alkaloids. Such a finding overcomes the problems associated with the use of a whole plant extract as discussed above and enables a safer and more effective alternative treatment option.

### BRIEF SUMMARY OF THE DISCLOSURE

In accordance with a first aspect of the present invention there is provided one or more mesembrine alkaloids for use in the treatment of diseases and conditions associated with anxiety and/or mood disorders.

Preferably the mesembrine alkaloid is mesembrenol and/or mesembranol. In one embodiment the mesembrine alkaloid is mesembrenol. In a further embodiment the mesembrine alkaloid is mesembranol.

Preferably the diseases or conditions associated with anxiety and/or mood disorders is selected from the group consisting of: generalised anxiety disorder; panic disorder; social anxiety disorder; phobia related disorder; major depressive disorder (MDD) or clinical depression; bipolar I disorder or manic depression; bipolar II disorder or hypomania; cyclothymic disorder; bipolar and related disorder due to another medical condition; depressive disorder due to another medical condition; substance or medication induced bipolar disorder; substance or medication induced depressive disorder; disruptive mood dysregulation disorder; persistent depressive disorder; and premenstrual dysphoric disorder.

In a further embodiment the diseases or condition associated with anxiety and/or mood disorders is treatment resistant.

Preferably the one or more mesembrine alkaloids is administered with one or more pharmaceutically acceptable excipients.

Preferably the one or more mesembrine alkaloids is formulated in a dosage form selected from a liquid, a lozenge, a fast-disintegrating tablet, a lyophilized preparation, a film, a spray, an aerosol, a sustained-release tablet or capsule, a modified release, a sustained relief, a tablet, a capsule a cream, an ointment, or a mucoadhesive.

Preferably the one or more mesembrine alkaloids is administered as a single daily dose. Alternatively, the mesembrine alkaloid is administered as multiple daily doses.

Preferably the one or more dose of mesembrine alkaloids is at least 0.001 mg of the compound.

In a further embodiment the one or more mesembrine alkaloids is administered with one or more additional drug products.

In accordance with a second aspect of the present invention there is provided a method of treating anxiety and/or mood disorders in a subject in need thereof, the method comprising administering to the subject a therapeutically effective amount one or more mesembrine alkaloids.

Preferably the one or more mesembrine alkaloids are present as highly purified mesembrine alkaloids. Alternatively, the one or more mesembrine alkaloids are present as synthetic mesembrine alkaloids.

Preferably the one or more mesembrine alkaloids are present as an extract derived from Mesembryanthemum tortuosum. More preferably the mesembrine alkaloids are present at greater than 50% of the total alkaloids present in the extract. More preferably the mesembrine alkaloids are present at greater than 60% of the total alkaloids present in the extract. More preferably the mesembrine alkaloids are present at greater than 70% of the total alkaloids present in the extract. More preferably the mesembrine alkaloids are present at greater than 80% of the total alkaloids present in the extract. More preferably the mesembrine alkaloids are present at greater than 90% of the total alkaloids present in the extract. More preferably the mesembrine alkaloids are present at greater than 95% of the total alkaloids present in the extract. More preferably the mesembrine alkaloids are present at greater than 97% of the total alkaloids present in the extract. More preferably the mesembrine alkaloids are present at greater than 98% of the total alkaloids present in the extract. More preferably the mesembrine alkaloids are present at greater than 99% of the total alkaloids present in the extract.

Alternatively, the one or more mesembrine alkaloids are present as a purified, isolated or semi-synthetic preparation.

When the mesembrine alkaloids of the invention are prepared synthetically the additional components present are not considered to be part of the active substance.

In a further embodiment the one or more mesembrine alkaloids used for the treatment of diseases and conditions associated with anxiety and/or mood disorders is not mesembrenone.

In human therapeutics, the physician will determine the dosage regimen that is most appropriate according to a preventive or curative treatment and according to the age, weight, stage of the disease and other factors specific to the subject to be treated. The compositions, in other embodiments, should provide a dosage of from about 0.0001 mg to about 70 mg of compound per kilogram of body weight per day. Dosage unit forms are prepared to provide from about 0.01 mg, 0.1 mg or 1 mg to about 500 mg, or about 1000 mg, and in some embodiments from about 10 mg to about 500 mg of the active ingredient or a combination of essential ingredients per dosage unit form.

The amount of active ingredient in the formulations provided herein, which will be effective in the prevention or treatment of a disorder or one or more symptoms thereof, will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above-described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### BRIEF SUMMARY OF THE DRAWINGS

The present invention is described with reference to the figures listed below:

FIG. 1 details the chronic mild unpredictable stress timeline.

FIG. 2 details effect of mesembrenol on female urine sniffing test.

FIG. 3 details the effect of mesembranol on female urine sniffing test.

FIG. 4 details the effect of mesembrenol on sucrose preference test.

FIG. 5 details the effect of mesembranol on sucrose preference test.

FIG. 6 details the effect of mesembrenol on the forced swim test.

FIG. 7 details the effect of mesembranol on the forced swim test.

FIG. 8 details the effect of mesembrenol on the elevated plus maze.

FIG. 9 details the effect of mesembranol on the elevated plus maze.

### LEGEND TO THE DRAWINGS

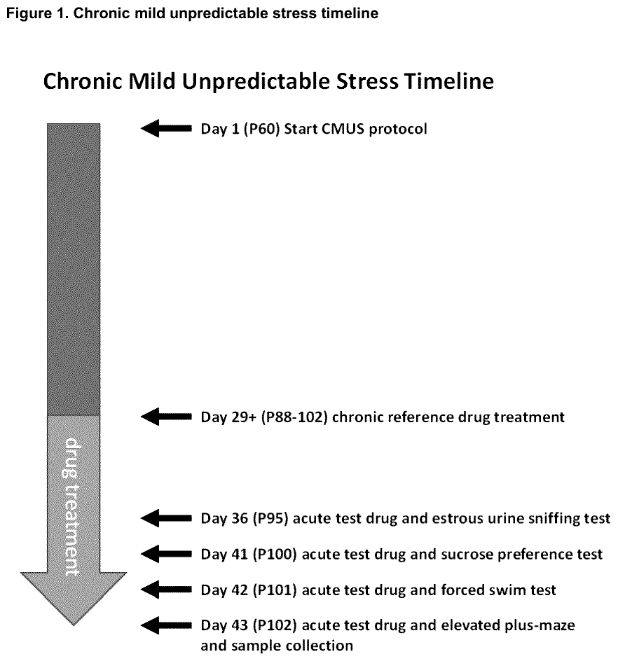

FIG. 1. Summary of the experimental timeline.

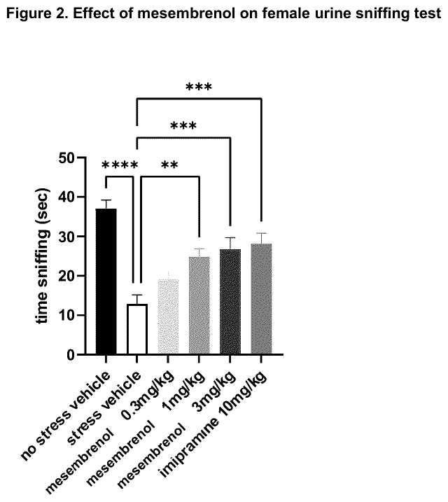

FIG. 2. Effect of mesembrenol (0.3, 1 and 3 mg/kg, i.p.) administered 60 minutes pre-test on preference to examine the female estrous urine treat following chronic mild unpredictable stress. Data represents total time (sec) engaged in sniffing and investigating the urine soaked cotton tip during a 3 minute trial period. Analysis by one-way ANOVA followed by multiple comparisons with Sidak's correction; **-****P<0.01-0.0001 vs stress vehicle (n=8).

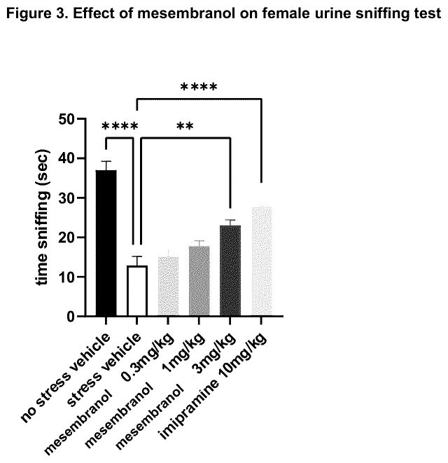

FIG. 3. Effect of mesembranol (0.3, 1 and 3 mg/kg, i.p.) administered 60 minutes pre-test on preference to examine the female estrous urine treat following chronic mild unpredictable stress. Data represents total time (sec) engaged in sniffing and investigating the urine soaked cotton tip during a 3 minute trial period. Analysis by one-way ANOVA followed by multiple comparisons with Sidak's correction; **-****P<0.01-0.0001 vs stress vehicle (n=8).

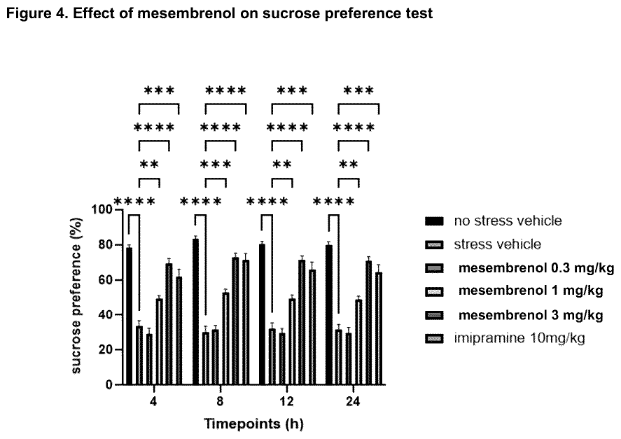

FIG. 4. Effect of mesembrenol (0.3, 1 and 3 mg/kg, i.p.) administered 30 minutes pre-test on sucrose preference following chronic mild unpredictable stress. Data represents cumulative % preference for sucrose solution over water at the 4, 8, 12 and 24 hour timepoints of the test period. Analysis by repeat measures two-way ANOVA followed by multiple comparisons with Dunnet's correction; *-****P<0.05-0.0001 vs stress vehicle (n=8).

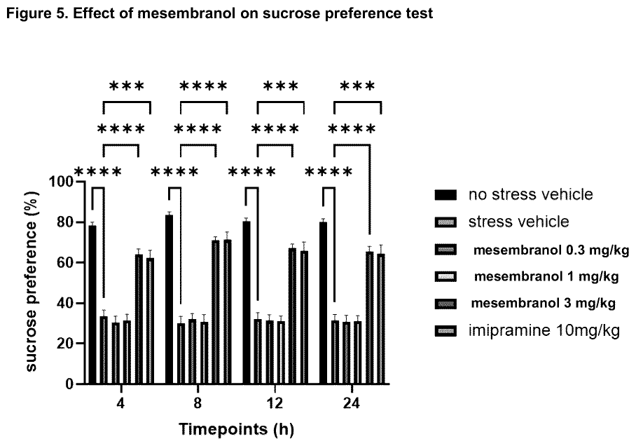

FIG. 5. Effect of mesembranol (0.3, 1 and 3 mg/kg, i.p.) administered 30 minutes pre-test on sucrose preference following chronic mild unpredictable stress. Data represents cumulative % preference for sucrose solution over water at the 4, 8, 12 and 24 hour timepoints of the test period. Analysis by two-way ANOVA followed by multiple comparisons with Sidak's correction; *-*P<0.001-0.0001 vs stress vehicle (n=8).

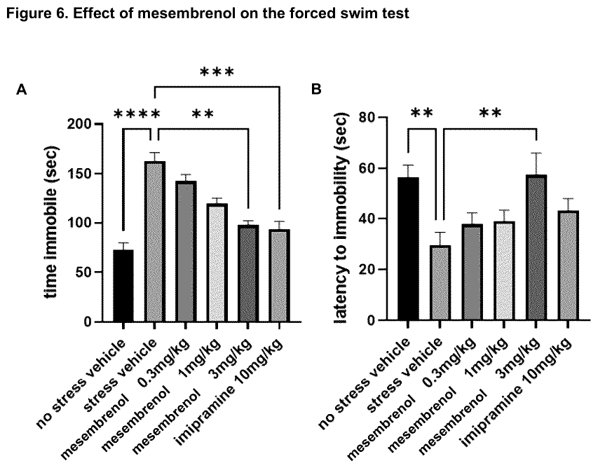

FIG. 6. Effect of mesembrenol (0.3, 1 and 3 mg/kg, i.p.) administered 30 minutes pre-test on chronic mild unpredictable stress induced depression phenotype in the forced swim test. Data represents (A) latency to immobility (sec) and (B) total time immobile in the four minute evaluation phase of the procedure. Analysis by one-way ANOVA followed by multiple comparisons with Sidak's and Dunn's correction; **-****P<0.01-0.0001 vs stress vehicle (n=8).

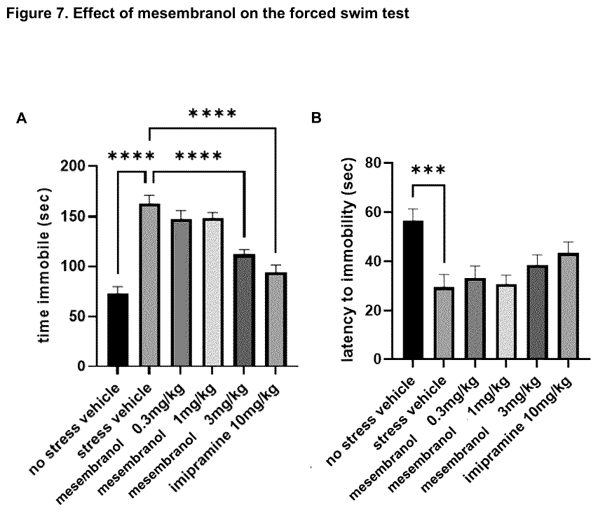

FIG. 7. Effect of mesembranol (0.3, 1 and 3 mg/kg, i.p.) administered 30 minutes pre-test on chronic mild unpredictable stress induced depression phenotype in the forced swim test. Data represents (A) latency to immobility (sec) and (B) total time immobile in the four minute evaluation phase of the procedure. Analysis by one-way ANOVA followed by multiple comparisons with Sidak's correction; ***-****P<0.001-0.0001 vs stress vehicle (n=8).

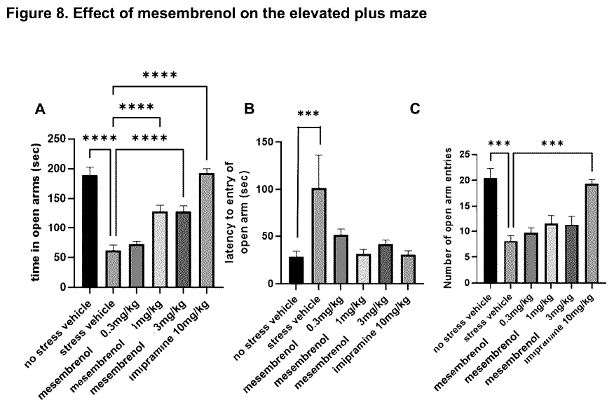

FIG. 8. Effect of mesembrenol (0.3, 1 and 3 mg/kg, i.p.) administered 30 minutes pre-test on chronic mild unpredictable stress induced anxiety phenotype in the elevated plus maze. Data represents (A) latency to open arm entry (sec), (B) number of open arm entries (a.u.) and (C) total time in open arms of the apparatus during the ten minute procedure. Analysis by one-way ANOVA followed by multiple comparisons with Dunn's and Sidak's correction; *-**** P<0.05-0.0001 vs stress vehicle (n=8).

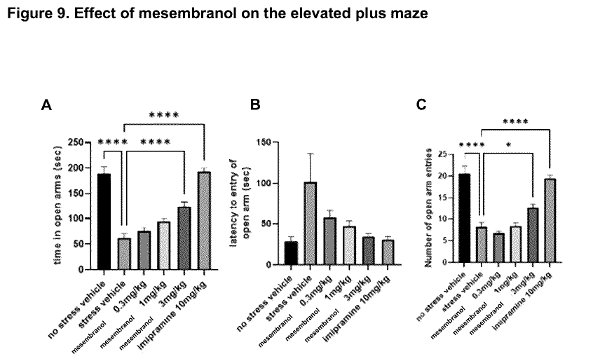

FIG. 9. Effect of mesembranol (0.3, 1 and 3 mg/kg, i.p.) administered 30 minutes pre-test on chronic mild unpredictable stress induced anxiety phenotype in the elevated plus maze. Data represents (A) latency to open arm entry (sec), (B) number of open arm entries (a.u.) and (C) total time in open arms of the apparatus during the ten minute procedure. Analysis by one-way ANOVA followed by multiple comparisons with Dunn's and Sidak's correction; *-**** P<0.05-0.0001 vs stress vehicle (n=8).

### Definitions

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

“Subject,” “individual” or “patient” is used interchangeably herein and refers to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof,). Treatment may also be considered to include preemptive or prophylactic administration to ameliorate, arrest or prevent the development of the disease or at least one of the clinical symptoms. Treatment can also refer to the lessening of the severity and/or the duration of one or more symptoms of a disease or disorder. In a further feature, the treatment rendered has lower potential for long term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to affect such treatment for the disease. The “therapeutically effective amount” will vary depending on the compound, the disease and its severity and the age, weight, adsorption, distribution, metabolism and excretion etc., of the patient to be treated.

The therapeutically effective amount of the mesembrine alkaloid of the invention is at least 0.001 mg. In some embodiments, the therapeutically effective amount of the mesembrine alkaloid is determined from pre-clinical animal studies. Guidance for the conversion of animal doses to human equivalent doses is provided in the FDA Guidance For Industry Document entitled “Estimating the Maximum Safe Starting Dose in Initial Clinical Trials for Therapeutics in Adult Healthy Volunteers” released July 2005, which is incorporated herein by reference. Table 1 from this document, which provides the conversion factors for converting between human and animal dosing, is excerpted below:

TABLE 1
Conversion of Animal Doses to Human Equivalent
Doses Based on Body Surface Area

| | |
|---|---|
| To Convert | To Convert Animal Dose |
| Animal Dose | in mg/kg to HED^{a} |
| in mg/kg | in mg/kg, Either: |

| | to Dose | Divide | Multiply |
|---|---|---|---|
| | in mg/m², | Animal | Animal |
| Species | Multiply by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula:
HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, thesus, and stumptail.

The human dose equivalent (HED) can be estimated using the following formula: HED=Animal dose (mg/kg) multiplied by (Animal Km/Human Km). The Km for a mouse is 3 and the Km for a human is 37.

Therefore, an effective dose in a mouse of 3 mg/kg would equate to a dose of 0.24 mg/kg in a human. This dose can be multiplied up to that of an average adult of 62 kg (global) or 80.7 kg (North America) or dosed at a level equivalent to the patient's bodyweight.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In embodiments, the pharmaceutically acceptable base addition salt is chosen from ammonium, potassium, sodium, calcium, and magnesium salts.

“Vehicle” refers to a diluent, excipient or carrier with which a compound is administered to a subject. In some embodiments, the vehicle is pharmaceutically acceptable.

“Anxiety” and/or “mood disorder” refers to any disease or condition which is caused by a patient suffering from anxiety or a mood disorder. In particular the diseases and conditions associated with anxiety and/or mood disorders of the invention include but are not limited to: generalised anxiety disorder; panic disorder; social anxiety disorder; phobia related disorder; major depressive disorder (MDD) or clinical depression; bipolar I disorder or manic depression; bipolar II disorder or hypomania; cyclothymic disorder; bipolar and related disorder due to another medical condition; depressive disorder due to another medical condition; substance or medication induced bipolar disorder; substance or medication induced depressive disorder; disruptive mood dysregulation disorder; persistent depressive disorder; and premenstrual dysphoric disorder.

The ICD-11 describes anxiety disorders as including the following: 6B00 Generalized Anxiety Disorder; 6B01 Panic Disorder; 6B02 Agoraphobia; 6B03 Specific Phobia; 6B04 Social Anxiety Disorder; 6B05 Separation Anxiety Disorder; 6B06 Selective Mutism and 6B0Y Other Specified Anxiety or Fear-Related Disorders.

The DSM-V describes mood disorders as including the following: Depressive Disorders, Disruptive Mood Dysregulation Disorder, Major Depressive Disorder, Single and Recurrent Episodes, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, and Unspecified Depressive Disorder.

The DSM-V describes anxiety disorders as including the following: Anxiety Disorders, Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical Condition, Other Specified Anxiety Disorder, and Unspecified Anxiety Disorder.

In some aspects of the invention the treatment of diseases and conditions is for the treatment of mood disorders and not the treatment of anxiety disorders.

“Treatment resistant” is defined as the failure of a disease or disorder to respond positively or significantly to treatment.

“Active ingredient” or “Active pharmaceutical ingredient” or “API” refers to the compounds of the invention.

“Highly purified mesembrine alkaloids” are defined as mesembrine alkaloids that have been extracted from the plant and purified to the extent that other mesembrine alkaloids and non-mesembrine alkaloid components that are co-extracted have been removed, such that the highly purified mesembrine alkaloid is greater than or equal to 95% (w/w) pure.

“Synthetic mesembrine alkaloids” are compounds that are structurally and chemically identical to the compound produced from the plant however have been manufactured using chemical means rather than by the plant.

The mesembrine alkaloids of the invention are detailed below:

| Compound structure | Full name |
|---|---|
| 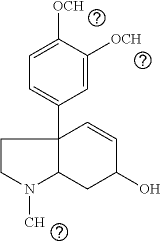 | Mesembrenol |
| 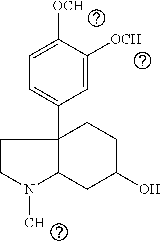 | Mesembranol |

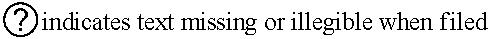

“Compounds of the invention” are one or more of the following compounds:
Mesembrenol, also known as ((3aS,7aS)-3a-(3,4-Dimethoxyphenyl)-1-methyl-2,3,4,5,7,7a-hexahydroindol-6-one), with a SMILES code:
O[C@@H]1C═C [C@@]2 (C3=CC═C(OC)C(OC)═C3) CCN(C) [C@@]2 ([H]) C1;
Mesembranol, also known as (3a-(3,4-dimethoxyphenyl)-1-methyl-3,4,5,6,7,7a-hexahydro-2H-indol-6-ol), with a SMILES code:
CN1CC [C@]2 ([C@@H]1C [C@@H](CC2)O) c3ccc(c(c3)OC)OC]

The compounds of the invention may be used as their salts or polymorph forms. Furthermore, the compounds may occur as an epimer, the cis-isomer, the trans-isomer or a racemate of the two. The compounds of the invention may be present or administered in the form of a prodrug of the active compound.

### DETAILED DESCRIPTION OF THE INVENTION

In some embodiments, the present invention provides the use of isolated mesembrine alkaloids in the prevention or treatment of anxiety and/or mood disorders. In a further embodiment the present invention provides the use of isolated mesembrine alkaloids in the prevention or treatment of anxiety and/or mood disorders methods, wherein the anxiety and/or mood disorders is comorbid with a second condition that is not a mood or anxiety disorder.

As used herein “mood disorders” broadly describe disorders in which a subject's emotional state or mood is distorted or inconsistent with their circumstances and interferes with their ability to function. As used herein, the terms “anxiety disorder,” refer to several different forms of abnormal, pathological anxiety, fears, and phobias encompassing psychiatric disorders of the nervous system based on stress, anxiety, or worry and not based on fact. Anxiety disorders include generalized anxiety disorder, panic disorder, phobias, social anxiety disorder, obsessive-compulsive disorder, post-traumatic stress disorder, and separation anxiety. Such disorders encompass anxiety, fears, and phobias related to or accompanying psychiatric conditions or disorders, specifically excluding anxiety caused by or related to trauma arising from ischemia, hemorrhagic insult (i.e., ischemic or hemorrhagic stroke), traumatic brain injury or resulting from underlying disease conditions accompanied by mental defects and/or cerebral or other neurodegeneration such as Alzheimer's disease, behavioral and psychological symptoms of dementia (BPSD), Huntington's disease, amyotrophic lateral sclerosis, Parkinson's disease, multiple sclerosis, schizophrenia, age-associated memory impairment, mild cognitive impairment, canine cognitive dysfunction syndrome, autism, autism spectrum disorder, Asperger syndrome, and Rett syndrome.

In some embodiments, the anxiety and/or mood disorder is selected from major depressive disorder (MDD), persistent depressive disorder, substance or medication induced depressive or anxiety disorder, depressive or anxiety disorder due to another medical condition, premenstrual dysphoric disorder (PMDD), depression, generalized anxiety disorder (GAD), separation anxiety disorder, specific phobia, social anxiety disorder, panic disorder, agoraphobia, behavioral and psychological symptoms of dementia (BPSD), other specified depressive or anxiety disorder, or, unspecified depressive or anxiety disorder.

In some embodiments, the anxiety and/or mood disorder is comorbid with a second disorder that is not an anxiety or mood disorder. For example, in patients diagnosed with neurological disorders including Huntington's disease, Parkinson's disease, Alzheimer's disease and dementia there is an increased probability that the patient will also present with symptoms of anxiety and/or mood disorder. Other conditions including heart disease, pancreatic disease, thyroid disease, cancer, infections and inflammatory bowel diseases such as Crohn's disease and ulcerative colitis may also often present with mood and/or anxiety disorders. Treatment of these patients is often limited to the treatment of the diagnosed disease rather than the comorbid conditions and as such there is a need for an effective treatment to ameliorate the mood and/or anxiety disorder symptoms that often occur with these conditions.

Further information relating to the anxiety and/or mood disorders of the invention are as follows:

### Behavioral and Psychological Symptoms of Dementia (BPSD)

BPSD is a combination of non-cognitive symptoms and behaviors that are comorbid in subjects with dementia. BPSD will develop in more than 90% of individuals diagnosed with dementia.

Dementia is the colloquial term that denotes the distinction of major neurocognitive disorder in the Diagnostic and Statistical Manual 5 edition (DSM-V). The presence of cognitive impairment is necessary and sufficient for a diagnosis of dementia. The associated neuropsychiatric symptoms of BPSD are prevalent and can significantly impact the prognosis and management of dementia. For this reason, DSM-V requires clinicians to specify whether BPSD is present and to specify the degree of severity. For example, a diagnosis of Alzheimer's dementia might be coded as “major neurocognitive disorder due to Alzheimer's disease, with behavioral disturbances, severe.”

The symptoms of BPSD are strongly correlated with the degree of function and cognitive impairment. Symptoms include, for example, impatience, mania, agitation, abnormal motor behavior, anxiety, exhilaration, irritability, depression, apathy, disinhibition, delusions, hallucinations, and changes in sleep or appetite.

Each of these symptoms must be assessed and treated independently of the underlying dementia. Psychotropic medications are frequently used to treat BPSD, although the side effect burden is high, and benefits are typically modest. As a result, few treatments exist to treat BPSD.

### Persistent Depressive Disorder

In some embodiments, the anxiety and/or mood disorder is persistent depressive disorder. Persistent depressive disorder (also referred to as dysthymic disorder) is a mild to moderate chronic depression. It involves a sad or dark mood most of the day, on most days, for two years or more.

The diagnostic criteria for persistent depressive disorder according to The Diagnostic and Statistical Manual of Mental Disorders, Fifth Edition (DSM-V) is as follows:

Depressed mood for most of the day, for more days than not, as indicated by subjective account or observation by others, for at least 2 years; presence while depressed of two or more of the following: poor appetite or overeating, insomnia or hypersomnia, low energy or fatigue, low self-esteem, poor concentration or difficulty making decisions, feelings of hopelessness.

During the 2 year period of the disturbance, the person has never been without symptoms from the above two criteria for more than 2 months at a time.

The disturbance is not better accounted for by major depressive disorder (MDD) or MDD in partial remission. Alternatively, criteria for MDD may be continuously present for 2 years, in which case patients should be given comorbid diagnoses of persistent depressive disorder and MDD.

There has never been a manic episode, a mixed episode, or a hypomanic episode and the criteria for cyclothymia have never been met.

The disturbance does not occur exclusively during the course of a chronic psychotic disorder or are not better explained by a psychotic disorder.

The disturbance is not due to the direct physiological effects of a substance (e.g., a drug of abuse or a medication) or a general medical condition.

The symptoms cause clinically significant distress or impairment in important areas of functioning.

Symptoms of persistent depressive disorder include loss of interest in daily activities, sadness, emptiness or feeling down, hopelessness, tiredness and lack of energy, low self-esteem, self-criticism or feeling incapable, trouble concentrating and trouble making decisions, irritability or excessive anger, decreased activity, effectiveness and productivity, avoidance of social activities, feelings of guilt and worries over the past, poor appetite or overeating, and sleep problems.

### Substance or Medication Induced Depressive or Anxiety Disorder

In some embodiments, the anxiety and/or mood disorder is a substance or medication induced depression or anxiety disorder. Substance or medication induced depression or anxiety disorders are characterized by anxiety or fear, sometimes accompanied by such physical symptoms as racing heart, breathless and shakiness, caused by the effects of a medication or psychoactive substance.

These symptoms may occur while the patient is under the influence of the drug (intoxication) or after use of the drug has stopped (withdrawal). Generalized anxiety, panic attacks or manifestations of phobia may be precipitated by substance use or withdrawal. Anxiety caused by the drug may persist as long as use continues, while withdrawal-related symptoms may first manifest themselves up to four weeks after cessation of use (Galanter and Kleber, 2008). Prolonged psychiatric symptoms, including anxiety and panic, can continue for up to six months, and have rarely been reported for years, after cessation of alcohol, benzodiazepine, opioid and occasionally antidepressant use (DeSoto, O'Donnell and DeSoto, 1989).

The DSM-V diagnostic criteria for substance/medication-induced anxiety disorder are those of the anxiety disorders, primarily anxiety and panic. Symptoms must develop during or within a month of use or intoxication, or within a month after withdrawal from a drug or substance known to cause anxiety. must not be ascribable to other anxiety disorders and must not be the result of delirium caused by the drug.

### Depressive or Anxiety Disorder Due to Another Medical Condition

In some embodiments, the anxiety and/or mood disorder is a depressive or anxiety disorder due to another medical condition. Depressive or anxiety disorders due to another medical condition are mood disorder diagnoses where there is a prominent and persistent period of depressed mood or markedly diminished interest/pleasure thought to be related to the direct physiological effects of another medical condition. Broadly speaking, however, the depression symptoms are similar to those found in other depressive disorders.

The DSM-V lists some of the comorbid pathologies associated with depressive disorder due to another medical condition. There is considerable evidence that Parkinson's disease can induce a state of depression (Ossowska and Lorenc-Koci, 2013). The percentage of Parkinson's disease patients affected by depression has not been completed and indisputably established; figures vary widely, from 4% up to 90% (Ossowska and Lorenc-Koci, 2013). A general consensus, however, is that at least 30% of Parkinson's disease patients have a depressive condition.

Patients with Huntington's disease frequently have a depressive disorder of some kind. Studies on the progression of depression in Huntington's disease have shown that it is negatively correlated with the increasing onset of motor symptoms (van Duijn et al., 2014). Depression is frequently observed in Huntington's disease patients (more than 50% of patients with Huntington's disease also have depressive conditions), but strikingly, it may develop years before any motor symptoms of Huntington's disease are present (Du et al., 2013). This makes depression due to Huntington's disease difficult to diagnose, since depression may be evident before any medical condition can be detected.

Stroke is often accompanied by depression (Espárrago Llorca et al., 2012), and in particular, it impacts over 30% of stroke patients, making it the most common psychiatric disorder that follows a stroke (termed “post-stroke depression”). Patients with post-stroke depression are at a higher risk of mortality than post-stroke patients who have no depression (at least 1 in 10 cases of post-stroke depression report suicide ideation), and hallmarks of post-stroke depression include social isolation and sleep complications.

Other medical conditions that result in clinical depression have been briefly discussed by the DSM-V; these include Cushing's disease, brain injury, and multiple sclerosis. Interestingly, at least one study has suggested that depression is a comorbid symptom of sickle cell anemia (Mahdi et al., 2010).

### Separation Anxiety Disorder

In some embodiments, the anxiety and/or mood disorder is separation anxiety disorder. Separation anxiety disorder is a DSM-V diagnosis assigned to individuals who have an unusually strong fear or anxiety to separating from people they feel a strong attachment to. The diagnosis is given only when the distress associated with the separation is unusual for an individual's developmental level, is prolonged, and severe. The need to stay in close proximity to caretakers can make it difficult for children with this disorder to go to school, stay at friends' houses or be in a room by themselves. In adults, it can make normal developmental activities like moving away from home, getting married, or being an independent person difficult.

The diagnostic criteria for separation anxiety disorder are as follows:

Developmentally inappropriate and excessive anxiety concerning separation from home or from those to whom the individual is attached characterized by: (1) recurrent excessive distress when anticipating or experiencing separation from home or from major attachment figures; (2) persistent and excessive worry about losing major attachment figures or about possible harm to them, such as illness, injury, disasters, or death; (3) persistent and excessive worry about experiencing an untoward event (e.g., getting lost, being kidnapped, having an accident, becoming ill) that causes separation from a major attachment figure; (4) persistent reluctance or refusal to go out, away from home, to school, to work, or elsewhere because of fear of separation; (5) persistent and excessive fear of or reluctance about being alone or without major attachment figures at home or in other settings; (6) persistent reluctance or refusal to sleep away from home or to go to sleep without being near a major attachment figure; (7) repeated nightmares involving the theme of separation; (8) repeated complaints of physical symptoms (such as headaches, stomach aches, nausea, or vomiting) when separation from major attachment figures occurs or is anticipated.

The fear, anxiety, or avoidance is persistent, lasting at least 4 weeks in children and adolescents and typically 6 months or more in adults.

The disturbance causes clinically significant distress or impairment in social, academic (occupational), or other important areas of functioning.

The disturbance is not better explained by another mental disorder, such as refusing to leave home because of excessive resistance to change in autism spectrum disorder; delusions or hallucinations concerning separation in psychotic disorders; refusal to go outside without a trusted companion in agoraphobia; worries about ill health or other harm befalling significant others in generalized anxiety disorder; or concerns about having an illness in illness anxiety disorder.

Symptoms of separation anxiety disorder include unusual distress at the discussion or experience of being parted from their attachment figure, excessive fears that harm will befall their attachment person, persistent worry of an unexpected event that could lead to separation from the attachment figure, refusal to leave the attachment figure, excessive fear of being alone, nightmares about separation, anxiety about sleeping and being separated from the attachment figure, physical complaints when separation is imminent.

### Specific Phobia

In some embodiments, the anxiety and/or mood disorder is a specific phobia. Specific phobia is a DSM-V diagnosis assigned to individuals who suffer from intense fear or anxiety when exposed to specific objects or situations. A type of anxiety disorder, specific phobias may present in response to a range of stimuli, from animals to medical procedures.

The diagnostic criteria for specific phobia is as follows: Marked fear or anxiety about a specific object or situation (e.g., flying, heights, animals, receiving an injection, seeing blood).

The phobic object or situation almost always provokes immediate fear or anxiety.

The fear or anxiety is out of proportion to the actual danger posed by the specific object or situation and to the sociocultural context.

The phobic object or situation is actively avoided or endured with intense fear or anxiety.

The fear, anxiety, or avoidance causes clinically significant distress or impairment in social, occupational, or other important areas of functioning.

The fear, anxiety, or avoidance is persistent, typically lasting for 6 months or more.

The disturbance is not better explained by the symptoms of another mental disorder, including fear, anxiety, and avoidance of situations associated with panic-like symptoms or other incapacitating symptoms (as in agoraphobia); objects or situations related to obsessions (as in obsessive-compulsive disorder); reminders of traumatic events (as in posttraumatic stress disorder); separation from home or attachment figures (as in separation anxiety disorder); or social situations (as in social anxiety disorder).

Examples of specific phobias include Animal Type (e.g., fear of spiders, insects, dogs), Natural Environment Type (e.g., fear of heights, storms, water), Blood-Injection-Injury Type (e.g., fear of needles, invasive medical procedures), and Situational Type (e.g., fear of airplanes, elevators, enclosed places).

Symptoms of specific phobia include increased heart rate (palpitations), dizziness or unsteadiness, nausea, sweating, shaking or trembling, and breathlessness. Someone suffering from a specific disorder will also display avoidance behavior, meaning that they take steps to avoid having to confront the object or situation at the center of their disorder.

### Social Anxiety Disorder

In some embodiments, the anxiety and/or mood disorder is social anxiety disorder. Social anxiety disorder, also known as social phobia, is an anxiety disorder involving discomfort around social interaction, and concern about being embarrassed and judged by others (NIH, 2014). This discomfort is experienced as fear and anxiety, and is accompanied by autonomic arousal, including diaphoresis, apnea, tremors, tachycardia, and nausea. It can range in severity to a discomfort which can be circumvented and adapted to, to a virtually disabling fear with infiltration into multiple areas of life. The discomfort that people with Social Anxiety Disorder experience can generalize to routine activities such as eating in front of others or using a public bathroom. Social anxiety can lead to isolation, and either absence of development or stagnation of social skills, which can intensify existing social anxiety.

The diagnostic criteria for social anxiety disorder include:

Marked fear or anxiety about one or more social situations in which the individual is exposed to possible scrutiny by others. Examples include social interactions (e.g., having a conversation, meeting unfamiliar people), being observed (e.g., eating or drinking), and performing in front of others (e.g., giving a speech). The individual fears that he or she will act in a way or show anxiety symptoms that will be negatively evaluated (i.e., will be humiliating or embarrassing; will lead to rejection or offend others). Note: In children, the anxiety must occur in peer settings and not just during interactions with adults.

Exposure to the feared social situation almost invariably provokes anxiety, which may take the form of a situationally bound or situationally predisposed panic attack. Note: In children, the anxiety may be expressed by crying, tantrums, freezing, clinging, shrinking, or failing to speak in social situations.

The fear or anxiety is out of proportion to the actual threat posed by the social situation and to the sociocultural context.

The social situations are avoided or endured with intense fear or anxiety.

The fear, anxiety, or avoidance causes clinically significant distress or impairment in social, occupational, or other important areas of functioning.

The fear, anxiety, or avoidance is persistent, typically lasting for 6 months or more.

The fear, anxiety, or avoidance is not attributable to the physiological effects of a substance (e.g., a drug of abuse, a medication) or another medical condition.

The fear, anxiety, or avoidance is not better explained by the symptoms of another mental disorder, such as panic disorder, body dysmorphic disorder, or autism spectrum disorder.

If another medical condition (e.g., Parkinson's disease, obesity, disfigurement from burns or injury) is present, the fear, anxiety, or avoidance is clearly unrelated or is excessive.

### Panic Disorder

In some embodiments, the anxiety and/or mood disorder is a panic disorder. Panic disorders are defined by the experience of unexpected panic attacks on a regular basis.

Diagnostic criteria of panic attacks include an abrupt surge of intense fear or intense discomfort that reaches a peak within minutes and during which time four or more of the following symptoms occur: (1) palpitations, pounding heart, or accelerated heart rate; (2) sweating; (3) trembling or shaking; (4) sensations of shortness of breath or smothering; (5) feeling of choking; (6) chest pain or discomfort; (7) nausea or abdominal distress; (8) feeling dizzy, unsteady, lightheaded, or faint; (9) derealization (feelings of unreality) or depersonalization (being detached from oneself); (10) fear of losing control or “going crazy”; (11) fear of dying; (12) paresthesias (numbness or tingling sensation); (13) chills or heat sensations.

### Agoraphobia

In some embodiments, the anxiety and/or mood disorder is agoraphobia. Agoraphobia is characterized anxiety about being in places or situations from which escape might be difficult (or embarrassing) or in which help may not be available in the event of having an unexpected or situationally predisposed panic attack or panic-like symptoms. Agoraphobic fears typically involve characteristic clusters of situations that include being outside the home alone; being in a crowd or standing in a line; being on a bridge; and traveling in a bus, train, or automobile. In severe cases, the individual becomes housebound, rarely leaving the house and, if so, only when accompanied.

The diagnostic criteria for agoraphobia include:

A marked fear or anxiety about two (or more) of the following five situations: (1) using public transportation; (2) being in open spaces; (3) being in enclosed spaces (e.g., shops, theaters, cinemas); (4) standing in line or being in a crowd; (5) being outside the home alone.

The situations are avoided (e.g., travel is restricted) or else are endured with marked distress or with anxiety about having a panic attack or panic-like symptoms or require the presence of a companion.

The agoraphobic situations almost always provoke fear or anxiety.

The fear or anxiety is out of proportion to the actual danger posed by the agoraphobic situations and to the sociocultural context.

The fear, anxiety, or avoidance is persistent, typically lasting 6 months or more.

The fear, anxiety, or avoidance causes clinically significant distress or impairment in important areas of functioning.

The anxiety or phobic avoidance is not better accounted for by another mental disorder.

### Other Specified Depressive or Anxiety Disorder and Unspecified Depressive or Anxiety Disorder

In some embodiments, the mood and/or anxiety disorder is a specified depressive or anxiety disorder or an unspecified depressive or anxiety disorder. Other specified depressive or anxiety disorder is a category of DSM-V diagnoses that applies to individuals who have symptoms characteristic of a depressive disorder (e.g.—major depressive disorder) or anxiety disorder, but do not meet the full criteria for any of them. Unspecified depressive or anxiety disorder are depressive or anxiety disorders that are impairing but do not fit any of the officially specified diagnoses.

### Premenstrual Dysphoric Disorder (PMDD)

In some embodiments, the anxiety and/or mood disorder is PMDD. PMDD is a symptom related to premenstrual phase (PMS) disorders. Premenstrual dysphoric disorder is considered as a subtype as well as the most severe form of premenstrual syndrome (Marvan ML, Cortes-Iniestra S. Women's beliefs about the prevalence of premenstrual syndrome and biases in recall of premenstrual changes. Health Psychol 2001; 20:276-80).

Selective serotonin reuptake inhibitors (SSRIs; e.g., fluoxetines, sertralines) and other psychotropic active ingredients (e.g., alprazolam) are current standard of care for symptomatic treatment of PMDD; however, treatment with these compounds can cause serious side effects. In addition, only a portion of the symptoms that constitute the PMDD image of disease can be mitigated with psychotropic active ingredients. In some embodiments, the methods of the present disclosure provide a pharmaceutical agent for the treatment of PMDD which avoids the drawbacks of pharmaceutical agents used to date.

According to the DSM-V, a woman with PMDD must have at least five premenstrual symptoms during the luteal phase, with at least one of the symptoms being an emotional or “core” symptom. The core symptoms must be irritability, anger, mood swings, tension or depression (and interfere with daily activities) and must be confirmed by a prospective daily rating for at least two cycles.

Symptoms of PMDD include mood swings, sudden feelings of sadness, increased sensitivity to rejection, episodes of anger or irritability, anxiety, panic attacks, depression, suicidal thoughts, loss of concentration, decreased interest in normal activities, fatigue, binge eating, headaches, hyper-insomnia, insomnia, breast tenderness, joint or muscle pain, a sensation of bloating, and weight gain.

### Generalized Anxiety Disorder (GAD)

In some embodiments, the anxiety and/or mood disorder is generalized anxiety disorder (GAD). GAD refers to a common chronic anxiety or mood disorder characterized by long-lasting anxiety that is not focused on any particular object or situation, i.e., it is unspecific or free-floating.

Diagnostic criteria for GAD include:
Excessive anxiety and worry (apprehensive expectation), occurring more days than not for at least 6 months, about a number of events or activities;
Difficulty controlling the anxiety and worry;
The anxiety and worry are associated with three or more of the following six symptoms, with at least some symptoms present for more days than not for the past 6 months: restlessness or feeling keyed up or on edge, being easily fatigued, difficulty concentrating or mind going blank, irritability, muscle tension, and sleep disturbance (difficulty falling or staying asleep, or restless unsatisfying sleep);
The focus of the anxiety and worry is not confined to features of an Axis I disorder, e.g., the anxiety or worry is not about having a panic attack (as in panic disorder), being embarrassed in public (as in social phobia), being contaminated (as in obsessive-compulsive disorder), being away from home or close relatives (as in separation anxiety disorder), gaining weight (as in anorexia nervosa), having multiple physical complaints (as in somatization disorder), or having a serious illness (as in hypochondriasis), and the anxiety and worry do not occur exclusively during posttraumatic stress disorder;
The anxiety, worry, or physical symptoms cause clinically significant distress or impairment in social, occupational, or other important areas of functioning; and
The disturbance is not due to the direct physiological effects of a substance or a general medical condition and does not occur exclusively during a mood disorder, a psychotic disorder, or a pervasive mental disorder.

The anxiety of sufferers from generalized anxiety may persist for long periods of time and is typically unrelated to particular situations or events; and sufferers may have felt anxious all their lives. Despite such constant anxiety, they may not realize that their worry is excessive, although they have trouble controlling their fears, or find that their worry interferes with their ability to function in social or occupational situations. Symptoms of GAD include excessive worry and/or excessive anxiety, restlessness, fatigue, loss of concentration, irritability, muscle tension, and sleep disturbance.

### Major Depressive Disorder (MDD)

In some embodiments, the mood and/or anxiety disorder is major depressive disorder (MDD). MDD is characterized by a persistently depressed mood and long-term loss of pleasure or interest in life, often with other symptoms such as disturbed sleep, feelings of guilt or inadequacy, and suicidal thoughts.

According to the DSM-V, the diagnostic criteria for MDD include:
Five or more of the following criteria (at least one includes 1 or 2): (1) Depressed mood-indicated by subjective report or observation by others (in children and adolescents, can be irritable mood); (2) loss of interest or pleasure in almost all activities-indicated by subjective report or observation by others; (3) significant (more than 5 percent in a month) unintentional weight loss/gain or decrease/increase in appetite (in children, failure to make expected weight gains); (4) sleep disturbance (insomnia or hypersomnia); (5) psychomotor changes (agitation or retardation) severe enough to be observable by others; (6) tiredness, fatigue, or low energy, or decreased efficiency with which routine tasks are completed; (7) a sense of worthlessness or excessive, inappropriate, or delusional guilt (not merely self-reproach or guilt about being sick); (8) impaired ability to think, concentrate, or make decisions-indicated by subjective report or observation by others; (9) recurrent thoughts of death (not just fear of dying), suicidal ideation, or suicide attempts.

The symptoms cause clinically significant distress or impairment in social, occupational, or other important areas of functioning.

The symptoms are not due to the direct physiological effects of a substance (e.g., drug abuse, a prescribed medication's side effects) or a medical condition (e.g., hypothyroidism).

There has never been a manic episode or hypomanic episode.

MDD is not better explained by schizophrenia spectrum or other psychotic disorders.

Treatment and Treatment Resistant Anxiety and/or Mood Disorders

There are different types of treatments available for anxiety and/or mood disorder such as therapy and medications. Treatments for anxiety and/or mood disorder have generally involved the administration of anti-anxiety and anti-depressant medications, opioid and non-opioid analgesics, and stimulants. These treatments have been limited and often ineffective as well as the cause of negative side effects. Side effects of such treatment may include, but are not limited to, overstimulation, insomnia, drug dependence, visual blurring, the urge to have more medication, feeling dysphoric or low, feeling hungry or eating more, binge eating, fatigue, amotivation, and poor concentration. In some embodiments, the methods provided herein do not result in the negative side effects associated with conventional treatments. Behavior therapy, cognitive behavior therapy and interpersonal therapy have all shown to be potentially beneficial the treatment of various mood and/or anxiety disorders.

In determining treatment, particularly for MDD, there are many types of depression scales that are used. One of the depression scales is a self-report scale called Beck Depression Inventory (BDI). Another scale is the Hamilton Depression Rating Scale (HAMD). HAMD is a clinical rating scale in which the patient is rated based on clinician observation. The Center for Epidemiologic Studies Depression Scale (CES-D) is a scale for depression symptoms that applies to the general population. This scale is typically used in research and not for self-reports. The PHQ-9 which stands for Patient-Health Questionnaire-9 questions, is a self-report as well. Finally, the Mood Disorder Questionnaire (MDQ) evaluates bipolar disorder.

In some embodiments, the anxiety and/or mood disorder is treatment resistant. The term “treatment-resistant” refers to the inability of standard treatment to reverse, alleviate, or inhibit the progression of the disorder to which such term applies, or one or more symptoms of the disorder. For example, in some clinical studies it is defined as patients with a principal DSM-IV diagnosis of generalized anxiety disorder who have not responded sufficiently after an adequate trial (4-8 weeks) of first-line anti-anxiety agents such as SSRIs, buspirone or a benzodiazepine. As used herein, the term also encompasses, depending on the condition of the patient, preventing the disorder, including preventing onset of the disorder or of any symptoms associated therewith, as well as reducing the severity of the disorder or any of its symptoms prior to onset, or to preventing a recurrence of a disorder.

The Examples below describe data for two specific, isolated mesembrine alkaloids, namely mesembrenol and mesembranol. These compounds were tested in four recognized animal models of anxiety and mood disorders. The models were undertaken using rodents which had been subjected to protocols which induce depression after the test animals were subjected to protocols which induce depression, in this case the chronic mild stress (CMS) paradigm was used.

Use of the CMS paradigm enables a better subject group to study as the condition of the animals is more like a human subject suffering from stress, anxiety or depression. Example 1 describes the CMS paradigm used to induce anxiety or depression in a test animal and Examples 2 to 5 detail the endpoint testing for the ability for the test compounds to treat anxiety or depression.

Example 6 details the pharmacokinetic data produced for the mesembrine alkaloids of the invention and Example 7 details the results from an in vitro pharmacology screen which determined the serotonin reuptake transporter (SERT) and Phosphodiesterase-4 (PDE-4) inhibition for mesembrenol and mesembranol.

Finally, Example 8 details the composition and purity of the chemically synthesized compounds mesembrenol and mesembranol used in the experiments.

### Example 1: Chronic Mild Stress Protocol

The chronic mild stress protocol induces chronic depression in a test animal. This was achieved by the protocol below.

### Methods

C57BI6J mice, will be housed in groups of 4, will be exposed to a variety of mild stressors for a period of four weeks.

The stressors will include wet bedding, 40° cage tilt, introduction of intruder mouse from different experimental cohort, food deprivation, water deprivation, overnight illumination, and presence of soiled rat bedding as an aversive odour. These stressors are randomly scheduled over each weekly period and repeated for the duration of the experiment. Each stressor is applied for a period of 8-16 hours.

Following 4 weeks of chronic unpredictable mild stress the animals will be randomly assigned to groups that will separately receive single daily injections of imipramine (10 mg/kg) for 7 days during which period the stress regimen will continue.

During the chronic stress regimen, animals will be housed in a room apart from control animals. All animals will be weighed daily.

This model was used to establish the potential anti-depressant-like and anxiolytic-like activity of two test compounds in a chronic model with relevance to depression in rodent species, chronic mild unpredictable stress.

### Detailed Protocol:

A cohort of depression-like phenotype in adult male C57B6J mice is produced by employing a four-week mild unpredictable stress protocol.

Following two weeks treatment with imipramine, or acute treatment with vehicle or the test compounds (mesembrenol and mesembranol), the reversal of stress induced depression-like behaviour was determined using a measure of anhedonia, female urine sniffing preference.

An additional readout was used to assess antidepressant-like activity of the test compounds using a further measure of anhedonia, the sucrose preference test.

The effect of treatment with the test compounds on chronic mild stress induced immobility in the forced swim test was tested to establish antidepressant-like activity.

Finally, the effect of the test compounds on chronic unpredictable mild stress anxiogenic phenotype in the elevated plus-maze to determine anxiolytic-like activity.

The test compounds were evaluated at three doses with comparison made to vehicle control, no stress control and the reference control imipramine (n=8).

### Drug Preparation

Imipramine was dissolved in 0.9% saline and administered by intraperitoneal injection, dose volume 1 ml/kg, at the same time daily for 14 to 15 days and 60 minutes prior to behavioural testing.

The test articles were formulated in 0.5% w/v (Hydroxypropyl)methyl cellulose (Sigma P8384 40-60cp, prepared in ultrapure water). The required volume of the HPMC to be added to the dry powder and gently mixed until fully dissolved. Formulation was a clear solution, stored in amber vials in freezer away from light.

The vehicle used was 0.5% methylcellulose.

### Results

The timeline for the CMS paradigm and the endpoint testing is detailed in FIG. 1.

The animals were deemed to have achieved a state of chronic depression prior to testing with the endpoint models as described in Examples 2 to 5 below.

### Conclusion

Animals were suitable for onwards testing.

### Example 2: Anhedonia Model—Female Urine Sniffing Test

The effects of chronic mild stress on hedonic behaviour will be assessed using a novel female urine sniffing test based on the natural interest of males in pheromones released from urine of the female mice in oestrus (Malkesman et al., 2010 Biol. Psychiatry 67:864).

### Methods

Initially, the test male mice are habituated to the presence of a dry cotton tipped applicator (2.2 mm×15 cm; VWR, Ireland) placed randomly on the floor of their home cage.

Animals are subsequently transferred to individual cages and exposed for a 3 minute period to a cotton tipped applicator which is dipped in water and attached to the side of the cage. The time each animal spends sniffing the water-impregnated cotton tipped applicator is scored manually by the observer who is blinded to group treatments.

Following a 45 minute interval in the absence of a cotton tipped applicator, an applicator dipped in the urine of a female mice in oestrus is presented and the time spent sniffing the urine-impregnated cotton bud is recorded over a 3 minute period.

Urine collected from several animals in oestrus will be combined to minimise inter-animal variation in pheromone content.

Testing is conducted under low level red light and behaviour is scored remotely via a video camera placed above the cage.

### Results

FIGS. 2 and 3 detail the results for mesembrenol and mesembranol respectively. As can be seen there was a significant reduction in time sniffing observed in stressed animals treated with vehicle relative to animals not subjected to stressors (**** p<0.0001) relative to vehicle.

There was a dose dependent increase in time sniffing with mesembrenol relative to vehicle. Specifically, there was a significant increase in time sniffing with mesembrenol at 1 mg/kg (** p<0.01) and mesembrenol at 3 mg/kg (*** p<0.001) relative to vehicle (FIG. 2).

There was also a significant increase in time sniffing observed with mesembranol at 3 mg/kg dose (** p<0.01) relative to vehicle (FIG. 3).

### Conclusion

A dose dependent effect was observed with both test compounds in the urine sniffing test and as such acute low doses appear to produce antidepressant-like effects in the estrous female urine sniffing test outcome in the CMS model.

### Example 3: Sucrose Preference Test

The sucrose preference test for rodents is based on the animal's natural preference for sweet tasting solutions, with the assumption that this preference is in proportion to the pleasure that the animal experiences when it consumes them. In general, this test measures the amount of a sweet-tasting solution that the animal ingests across a fixed period.

### Methods

During this test, mice are housed individually and provided, for 24 hours, a free choice between two bottles, one with 1% sucrose solution and another with filtered tap water.

To prevent possible effects of side preference in drinking behaviour, the position of the bottles is swapped after 12 hours. The consumption of water and sucrose solution is estimated simultaneously in control and experimental groups by weighing the bottles. The sucrose intake is calculated as an amount of consumed sucrose in mg per gram body weight. The preference for sucrose is calculated as a percentage of consumed sucrose solution of the total amount of liquid drunk.

A decrease of sucrose preference below 65%, is taken as the criterion for anhedonia. This criterion is based on the fact that control mice do not exhibit this decreased preference for sucrose. In addition, mice with a lower than 65% sucrose preference in other models display features of anhedonia and depression, such as an increased threshold of intracranial self-stimulation and sleep disturbances.

The effect of 2 weeks treatment with imipramine or the acute effect of the test compound or vehicle is determined over this 24 hour period.

### Results

An increase in sucrose preference depicts an hedonic (antidepressant) like effect.

FIGS. 4 and 5 detail the results for mesembrenol and mesembranol respectively. As can be seen there was a significant reduction in preference for sucrose in stressed animals treated with vehicle relative to animals not subjected to stressors treated with vehicle across all time points tested.

There was a dose dependent increase in sucrose preference with mesembrenol relative to the stress vehicle group across all time points tested.

Specifically, there was a significant increase in sucrose preference at 1 mg/kg and 3 mg/kg relative to the stress vehicle group across all time points tested (FIG. 4).

There was also a statistically significant increase in sucrose preference with mesembranol at 3 mg/kg relative to the stress vehicle group at all timepoints tested (FIG. 5).

### Conclusion

Both test compounds at acute low doses appear to produce hedonic (antidepressant)-like in the sucrose preference test outcome in the CMS model.

### Example 4: Forced Swim Test

The forced swim test is a standard model for examination of potential antidepressant-like activity and represents futility, despair, and motivation behavioural domains.

### Methods

Following the 24 hour sucrose preference test, animals are treated again with the test compound, imipramine or vehicle. Animals are individually placed in clear Plexiglas cylinders (25 cm high; 10 cm in diameter) which are filled to a depth of 15 cm with water at 22-25° C.

Testing is conducted under low level red light and behaviour recorded over a 6 minute period using a video camera placed above the cylinder. During the test the animals are placed in the cylinder and allowed to habituate for two minutes before the cumulative duration of time spent immobile is determined over the following 4 minutes. Latency to immobility is recorded.

Movements necessary for the animal to maintain its head above water will not be scored as activity.

### Results

A reduction in immobility time or an increase in latency to immobility depicts an antidepressant-like effect.

FIGS. 6 and 7 detail the results for mesembrenol and mesembranol respectively. As can be seen there was a significant increase in immobility time and significant reduction in latency to immobility observed in stressed animals treated with vehicle relative to animals not subjected to stressors treated with vehicle.

There was a significant reduction in immobility time and significant increase in latency to immobility with mesembrenol at 3 mg/kg relative to vehicle.

Furthermore, there was a significant reduction in immobility time with no changes in latency to immobility with mesembranol at 3 mg/kg relative to vehicle.

### Conclusion

Both test compounds at acute low doses appear to produce antidepressant-like effects in the forced swim test outcome in the CMS model.

### Example 5: Elevated Plus Maze

The elevated X-maze has strong claims to validity as an animal model of anxiety and is of great utility in evaluating putative anxiety-modulating drugs (Handley and McBlane, 1993. J. Pharm. Tox. Methods 29:129). The test is based on rodents' natural fear of heights and open spaces.

### Methods

The apparatus consists of 4 raised flat runways extending from a central platform raised 1 m above the floor of a 2 m wide water maze (prevents escape in the rare event of an animal falling from the apparatus). Two arms have enclosed sides while the others are open.

The mouse version of the apparatus employs maze arms of 30 cm length and 6 cm width, where the enclosed arms have walls 30 cm high. The room is lit with low level indirect lighting.

Animals are allowed to freely explore the apparatus over a 10 minute trial with remote video analysis capturing the pattern of animal movement. The time animals spend exploring the open arms of the maze during the trial is inversely related to the level of stress/anxiety felt by the animal.

Endpoints recorded are the latency (sec) to enter open arms of the maze, total number of open arm entries (a.u.) and total time (sec) spent in the open arms of the plus-maze.

### Results

An increase in time spent in the open arms, reduction in latency to enter the open arms or an increase in open arm entries depicts an anxiolytic-like effect.

FIGS. 8 and 9 detail the results for mesembrenol and mesembranol respectively. As can be seen there was a significant reduction in the time spent in the open arms, a significant increase in the latency to entry of open arms, a significant increase in the latency to entry of open arms and significant reduction in the number of open arm entries observed in stressed animals treated with vehicle relative to animals not subjected to stressors treated with vehicle.

There was a significant increase in the time spent in the open arms with mesembrenol at 1 mg/kg and 3 mg/kg relative to vehicle. There was no change in number of open arm entries between mesembrenol and the vehicle group.

Furthermore, there was a significant increase in the time spent in the open arms and number of open arm entries with mesembranol at 3 mg/kg relative to vehicle.

### Conclusion

Both test compounds at acute low doses appear anxiolytic-like in the elevated plus maze outcome in the CMS model.

### Example 6: Pharmacokinetic Study in Mice

### Methods:

Groups of male C57BI/6J mice received a single administration of test compound (10 mg/kg; i.p.) in methylcellulose (0.5% w/v).

Three mice from each dose group were subject to cardiac puncture under general anaesthesia at 0.25, 0.50, 1.00, 2.00, 4.00, 7.00 and 24.0 hours post-dose and plasma samples generated for application of standard LC-MS/MS bioanalytical methods.

Quantitation of compound concentration was derived from reference calibration data. Pharmacokinetic data were derived from serial plasma concentrations.

### Results:

Table 1 details the PK parameters measures for mesembrenol and mesembranol.

TABLE 1
Summary of PK parameters

| | Cₘₐₓ | Tₘₐₓ | AUC0-inf | t_{1/2} |
|---|---|---|---|---|
| Compound | (ng/mL) | (hr) | (ng · hr/mL) | (hr) |
| Mesembrenol | 503 | 0.267 | 352 | 0.438 |
| Mesembranol | 834 | 0.300 | 649 | 0.363 |

### Conclusion:

Both mesembrine alkaloids demonstrated a short Tmax at around 18 minutes and relatively short half-lives of less than half an hour. These effects are indicative of medications that will take effect quickly and be cleared from the body in a short space of time also.

Mesembranol had a higher Cₘₐₓ and AUC than mesembrenol. This finding was surprising given that data presented in Examples 2 to 5 demonstrate that mesembrenol displays better efficacy than mesembranol at lower doses in the animal models of anxiety and mood disorders.

### Example 7: In Vitro Pharmacology Inhibition Assays

### SERT Assay (Compounds of Formula I):

Test compounds (conc range), reference compound or vehicle control were incubated for 180 min at room temperature with CHO cells stably transfected with the human serotonin transporter (5×10³ cells/well) with 0.15 μM [3H] serotonin in the presence or absence of the test or reference compound in buffer containing (in mM); Tris/HCl (pH 7.4) (5), HEPES/Tris (7.5), NaCl (120), KCl (5.4), CaCl₂) (1.2), MgSO4 (1.2), Glucose (5) and ascorbic acid (1). Following incubation, [3H] serotonin uptake was quantified by a standard scintillation counting method.

### Pde4-B1 & Pde4-D2 Assays:

Test compounds (conc range), reference compound or vehicle control were added to assay buffer containing (in mM); Tris/HCl, pH 7.4 (40), MgCl2 (8) and EGTA/NaOH (1.7), containing 450 nM cAMP (450 nM) and 0.25 μCi (PDE-4B1) or 0.0125 μCi (PDE-4D2) [³H] CAMP. 20 mins after addition of human recombinant PDE-4B1 (1.2 IEU) or PDE-4D2 (1.5 IEU), SPA beads were added and incubated at 22° C. for a further 30 mins. [³H]5′AMP was quantified by a standard scintillation counting method.

### Statistical Analysis:

Data were expressed as the mean of two technical replicates for each data point, and percent inhibition calculated relative to maximum inhibition observed in the presence of the reference comparator rolipram for PDE-4 assays, and imipramine for serotonin assays.

### Results

Table 2 below details the IC₅₀ values derived for mesembrenol and mesembranol in the in vitro assays. Also displayed are the reference comparator data.

TABLE 2
Potency determinations for compounds at SERT and PDE-4

| | IC₅₀ (nM) | | |
|---|---|---|---|
| Compound | SERT | PDE-4B1 | PDE-4D2 |
| Mesembrenol | 462 | 27600 | 3910 |
| Mesembranol | 143 | 8410 | 9840 |
| Imipramine | — | 1600 | 144-300 |
| Rolipram | 23.2 | — | — |

SERT - serotonin transporter; PDE-4 - Phosphodiesterase-4.

### Conclusion:

The in vitro data demonstrates the compounds are unlikely to be inhibitors of either serotonin transporter (SERT) or phosphodiesterase-4 (PDE-4).

Inhibition of SERT is demonstrated by nearly all antidepressants to varying degrees. Inhibiting SERT increases the amount serotonin available in the synapse, which in turn leads to downstream cellular and molecular adaptations that are thought to mediate the efficacy of medications which are used as antidepressants.

Phosphodiesterase-4 (PDE-4) is mainly present in immune cells, epithelial cells, and brain cells. It manifests as an intracellular non-receptor enzyme that modulates inflammation and epithelial integrity. A compound's ability to inhibit PDE-4 is suggestive that they may serve as a promising therapeutic target for the treatment of diverse pulmonary, dermatological, and severe neurological diseases.

These data are surprising as the data in the animal studies in Examples 1 to 5 clearly demonstrate the ability of the mesembrine alkaloids of the invention to be effective in the treatment of anxiety and mood disorders. As such it is clear that they are able to elicit their effects through a different mechanism of action than through SERT unlike the common antidepressants on the market.

### Example 8: Certificates of Analysis

### Methods:

The compounds of the invention mesembrenol and mesembranol used in all of the experiments described herein are detailed below.

### Results:

Table 3 below details the analysis of the two compounds.

TABLE 3
Analytical details of the compounds used in the invention

| | | Retention time (min) | | |
|---|---|---|---|---|
| Compound | Purity | GC-MS | HPLC | Description |
| Mesembrenol | >97% | 8.36 | 9.3 | Slightly |
| | | | | brown solid |
| Mesembranol | >97% | 8.38 | 13.4 | Yellowish to |
| | | | | brown syrup |

### Conclusion:

Both compounds used in the experiments presented in the patent application were of a very high purity of greater than 97%.

### Overall Conclusion

The series of experiments detailed in the present application strongly demonstrate the ability of both compounds mesembrenol and mesembranol to be useful in the treatment of diseases and conditions associated with anxiety and/or mood disorders.

Contrary to the data generated by Maphanga et al., 2022 which showed particular efficacy of mesembrenone and mesembranol in a zebrafish model of anxiety, our data suggested that mesembrenol was more efficacious and was able to exert it's effects at a lower dose than mesembranol.

Also contrary to the Maphanga et al., 2022 paper where a predictive in silico model was used to show that the compounds would be inhibitors of SERT and act in a similar manner to approved antidepressants, our data demonstrates that the compounds are unable to inhibit SERT in an in vitro model raising questions over the purity of the compounds used in the paper.

The certificates of analysis of the compounds used in the experiments of this application demonstrate that the mesembrenol and mesembranol were greater than 97% whereas the compounds in the Maphanga paper are described as having a purity of greater than 85%. It would appear that the impurities present in the compositions used in Maphanga may be having an impact on both the efficacy and binding of the compounds and as such cannot be treated as equivalent.

1. One or more mesembrine alkaloids for use in the treatment of diseases and conditions associated with anxiety and/or mood disorders.

2. One or more mesembrine alkaloids for use according to claim 1, wherein the mesembrine alkaloid is mesembrenol and/or mesembranol.

3. One or more mesembrine alkaloids for use according to claim 1, wherein the mesembrine alkaloid is mesembrenol.

4. One or more mesembrine alkaloids for use according to claim 1, wherein the mesembrine alkaloid is mesembranol.

5. One or more mesembrine alkaloids for use according to any of the preceding claims, wherein the diseases or conditions associated with anxiety and/or mood disorders is selected from the group consisting of: generalised anxiety disorder; panic disorder; social anxiety disorder; phobia related disorder; major depressive disorder (MDD) or clinical depression; bipolar I disorder or manic depression; bipolar II disorder or hypomania; cyclothymic disorder; bipolar and related disorder due to another medical condition; depressive disorder due to another medical condition; substance or medication induced bipolar disorder; substance or medication induced depressive disorder; disruptive mood dysregulation disorder; persistent depressive disorder; and premenstrual dysphoric disorder.

6. One or more mesembrine alkaloids for use according to any of the preceding claims, wherein the diseases or condition associated with anxiety and/or mood disorders is treatment resistant.

7. One or more mesembrine alkaloids for use according to any of the preceding claims, wherein the mesembrine alkaloid is administered with one or more pharmaceutically acceptable excipients.

8. One or more mesembrine alkaloids for use according to any of the preceding claims, wherein the mesembrine alkaloid is formulated in a dosage form selected from a liquid, a lozenge, a fast-disintegrating tablet, a lyophilized preparation, a film, a spray, an aerosol, a sustained-release tablet or capsule, a modified release, a sustained relief, a tablet, a capsule a cream, an ointment, or a mucoadhesive.

9. One or more mesembrine alkaloids for use according to any of the preceding claims, wherein the mesembrine alkaloid is administered as a single daily dose.

10. One or more mesembrine alkaloids for use according to any of the preceding claims, wherein the mesembrine alkaloid is administered as multiple daily doses.

11. One or more mesembrine alkaloids or use according to claim 9 or 10, wherein each dose comprises at least 0.001 mg of the mesembrine alkaloid.

12. One or more mesembrine alkaloids for use according to any of the preceding claims, wherein the mesembrine alkaloid is administered with one or more additional drug products.

13. A method of treating anxiety and/or mood disorders in a subject in need thereof, the method comprising administering to the subject a therapeutically effective amount one or more mesembrine alkaloids.
